(19) United States
(12) Patent Application Publication
(10) Pub. No.: US 2008/0311634 A1
(43) Pub. Date: Dec. 18, 2008

(54) Flavonoids-rich tissue from Belamcanda chinensis and methods for culturing the same

(76) Inventors: Chin-Wen Ho, Taipei (TW); Tin-Hui Chu, Taipei (TW)

Correspondence Address: ANDREWS KURTH LLP;Intellectual Property Department, Suite 1100, 1350 I Street, N.W., Washington, DC, 20005, US

(21) Appl. No.: 11/812,075

(22) Filed: Jun. 14, 2007

(51) Int. Cl. C12P 17/06; G01N 30/02

(52) U.S. Cl. 435/125; 436/161

(57) ABSTRACT

The present invention provides an in vitro flavonoid-rich tissue of Belamcanda chinensis, which is produced in a tissue culture system using a B. chinensis tissue capable of proliferation, such as a seed, an embryo of said seed, a root, a leaf, a base of a leaf, or a young inflorescence of B. chinensis. The preferred in vitro flavonoid-rich tissue is a callus tissue or an fast-proliferated roots of B. chinensis which contain a very high content of flavonoid as compared to the wild type B. chinensis. The present invention further provides a method for inducing the formation of the callus tissue and the fast-proliferated roots of B. chinensis. It also provides a method for extracting the flavonoids and a quantitative method for determining the amount of total flavonoids from the in vitro flavonoid-rich tissue.

### FIELD OF THE INVENTION

The present invention relates to an in vitro flavonoid-rich tissue of Belamcanda chinensis, which is produced in a tissue culture system using a B. chinensis tissue capable of proliferation, such as a seed, an embryo of the seed, a root, a leaf, a base of a leaf, or a young inflorescence of B. chinensis. The in vitro flavonoid-rich tissue of B. chinensis is preferably a callus tissue or a fast-proliferated root which contains a very high content of flavonoids, as compared to the wild type B. chinensis. The present invention further provides a method for inducing the formation of the in vitro flavonoid-rich tissue from B. chinensis. It also provides a method for extracting the flavonoids from and a quantitative method for determining the amount of total flavonoids in the in vitro flavonoid-rich tissue.

### BACKGROUND OF THE INVENTION

Phytochemicals are substances that plants naturally produce to protect themselves against bacteria, viruses, and fungi. There has been a lot of interest in phytochemicals recently because many of them can help to slow the aging process and reduce the risk for cancer, heart disease, and other chronic health conditions.

More than 900 different phytochemicals have been found in plant foods, with others still to be discovered. Fruits, vegetables, whole grains, soy and nuts are all sources of these disease-fighting substances. Phytochemicals are usually related to plant pigments, so fruits and vegetables with bright colors (yellow, orange, red, blue, purple, green) contain the most.

Flavonoids are a group of chemicals that belong to the phytochemicals family. They are mostly plant secondary metabolites. According to the IUPAC nomenclature, they can be classified into: flavonoids, derived from the 2-phenylchromen-4-one(2-phenyl-1,4-benzopyrone) structure; isoflavonoids, derived from the 3-phenylchromen-4-one (3-phenyl-1,4-benzopyrone) structure; and neoflavonoids, derived from the 4-phenylcoumarine (4-phenyl-1,2-benzopyrone) structure. All flavonoids share the molecular structure of the flavone backbone (2-phenyl-1,4-benzopyrone), as shown below:

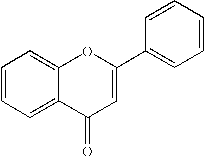

Flavonoids have long been recognized to possess anti-inflammatory, antioxidant, antiallergic, hepatoprotective, antithrombotic, antiviral, and anticarcinogenic activities.

Flavonoids are typical phenolic compounds and, therefore, act as potent metal chelators and free radical scavengers. They are powerful chain-breaking antioxidants. Flavonoids display a remarkable array of biochemical and pharmacological actions, some of which suggest that certain members of this group of compounds may significantly affect the function of various mammalian cellular systems. Reports indicate that plant flavonoids cause the activation of bacterial (Rhizobium) modulation genes involved in control of nitrogen fixation, which suggests important relationships between particular flavonoids and the activation and expression of mammalian genes (See e.g., Midddledton et al. Pharmacological Reviews, 2000, 52:673-751).

B. chinensis, often referred to as blackberry lily, is a common horticulture plant that derives its name from the clusters of shiny black seeds exposed when seed capsules split open. Even though it is called a lily, B. chinensis actually belongs to the Iridaceae family. B. chinensis is propagated by division of rhizomes or by seed. Seeds need a 4 to 6 week cold stratification period. The natural growing period of B. chinensis (i.e., from seeding to harvest), however, is about two to three years.

B. chinensis has been known for its medicinal effects for the treatment of mumps, hepatitis, and gums ache disease. It in fact has been used as an herb (i.e., the rhizome of B. chinensis) in traditional Chinese medicine for many decades. Additionally, the seeds of B. chinensis have been known to be a common source for flavonoids. In fact, B. chinensis is most famous as the primary natural source for a particular type of flavonoids, tectorigenin. Due to its usefulness in producing flavonoids and its notoriously long harvest period, there exists a need for finding a way to grow B. chinensis with high flavonoid content in a short period of time.

### SUMMARY OF THE INVENTION

The present invention provides an in vitro flavonoid-rich tissue of Belamcanda chinensis. The in vitro flavonoid-rich tissue is produced in a tissue culture system and is characterized by its elevated total flavonoid content, i.e., the flavonoid content is about 9 times of that of the wild type B. chinensis when cinnamic acid is used as the standard for measurement or about 3 times of that of the wild type B. chinensis when ψ-tectorigenin is used as the standard for measurement.

The preferred in vitro flavonoid-rich tissue is a callus tissue or fast-proliferated roots of B. chinensis. The in vitro flavonoid-rich tissue of B. chinensis is cultured from a B. chinensis tissue capable of proliferation, such as a seed, an embryo of the seed, a root, a leaf, a base of a leaf, or a young inflorescence of B. chinensis.

The tissue culture system is a solid or liquid flask system. The preferred system for callus induction, somatic embryogenesis, and plantlet regeneration is the solid flask system. The tissue culture system contains a culture medium, which primarily contains a salt medium and a carbohydrate. The salt medium is preferred to be a Murashige and Skoog basic salt medium (MS medium), which contains primarily the following salts: sodium, potassium, nitrate, ammonium, magnesium, sulfate, calcium, iron, chloride, phosphate, manganese, iodine, borate, zinc, copper, molybdenum, cobalt, or a mixture thereof. The preferred carbohydrate is myo-inositol or sucrose or a mixture thereof. The culture medium is preferred to be maintained at a pH of about 5.0 to 7.0

Optionally, the culture medium can further contain a plant growth regulator, such as indole-3-acetic acid (IAA), 2-4-dichlorophenoxyacetic acid (2,4-D), α-naphthaleneacetic acid (NAA), 6-benzyl-aminopurine (BA), kinetin, or a mixture thereof.

Additionally, the culture medium can further optionally contain a vitamin, such as thiamine HCl, pyridoxine HCl, nicotinic acid, or a mixture thereof.

The present invention also provides a method for preparing the in vitro flavonoid-rich tissue of B. chinensis. The method comprises: (1) inoculating a B. chinensis tissue in a culture medium of a tissue culture system; and (2) growing the B. chinensis tissue in the tissue culture system for a sufficient amount of time to allow the in vitro flavonoid-rich tissue tissue to form. The B. chinensis tissue to be used for the inoculation is one that is capable of proliferation.

The culture medium is maintained at about 20° C. to 30° C.

The B. chinensis tissue which is suitable for use in this preparation is preferred to be a seed, an embryo of the seed, a root, a leaf, a base of a leaf, or a young inflorescence of B. chinensis. The sufficient amount of time for the callus tissue to be ready for subculture or passage is about 2 to 8 weeks, preferably 4-5 weeks.

The culture medium contains primarily a salt medium, and a carbohydrate. The preferred salt medium for the tissue culture system is a Murashige and Skoog basic salt medium (MS medium), which comprises sodium, potassium, nitrate, ammonium, magnesium, sulfate, calcium, iron, chloride, phosphate, manganese, iodine, borate, zinc, copper, molybdenum, cobalt, or a mixture thereof. The preferred carbohydrate is myo-inositol or sucrose or a mixture thereof.

Optionally, the culture medium further comprises a plant growth regulator, such as indole-3-acetic acid, 2-4-dichlorophenoxyacetic acid, α-naphthaleneacetic acid, 6-benzyl-aminopurine, kinetin, or a mixture thereof. The culture medium can also contain a vitamin, such as thiamine HCl, pyridoxine HCl, nicotinic acid, or a mixture thereof. The culture medium is preferred to have a pH of about 5 to 7.

The present invention further provides a method for extracting the flavonoids from the in vitro flavonoid-rich tissue of B. chinensis. The method comprises the steps of: (1) drying the in vitro flavonoid-rich tissue of B. chinensis to obtain a dried flavonoid-rich tissue; (2) grinding the dried tissue; (3) adding an alcohol to the dried flavonoid-rich tissue to form a suspension; (4) heating the suspension to form a heated suspension; and (5) filtering the heated suspension after the heated suspension has cooled off to collect an filtrate which contains the flavonoids.

The dried flavonoid-rich tissue is obtained by subjecting said flavonoid-rich tissue to freeze-drying. The suspension is preferred to be heated at about 50-70° C., and, preferably, with vibration. The vibration is preferred to be generated by an ultrasonic wave. The alcohol which is added to the dried flavonoid-rich tissue is preferred to be methanol or ethanol. The filtrate is collected by passing said heated suspension through a Whatman® No. 1 filter.

Finally, the present invention provides methods for determining the total amount of flavonoids and tectorigenin extracted from the in vitro flavoid-rich tissue of B. chinensis, and for determining the various types of flavonoids produced in the in vitro flavoid-rich tissue. There are two methods used for determining the total amount of flavonoids. The first method involves the use of the cinnamic acid, which is a precursor of flavonoids, as a standard. The method comprises: (1) measuring the tissue extract in a spectrophotometer at an absorbance of 367 nm wavelength to obtain a sample absorbance value; and (2) comparing the sample absorbance value to a standard absorbance value. The tissue extract is pretreated with AlCl₃. The second method involves the use of the ψ-tectorigenin as a standard. The method comprises: (1) measuring the tissue extract in a spectrophotometer at an absorbance of 510 nm wavelength to obtain a sample absorbance value; and (2) comparing the sample absorbance value to a standard absorbance value. The tissue extract is pretreated with NaNO₂ and AlCl₃.

The various types of flavonoids extracted from the in vitro flavonoid-rich tissue of B. chinensis are analyzed by injecting the filtrate to a column of a high performance liquid chromatography (HPLC); and recording the elution profile of the HPLC at an absorbance of 265 nm. The results demonstrate that there was a change of the flavonoid profile in the in vitro flavonoid-rich tissue of B. chinensis, as compared to the wild type rhizome of B. chinensis, i.e., a much greater proportion of the flavonoid precursors than the isoflavonoids, by comparing the HPLC elution profile of the extract from the in vitro flavonoid-rich tissue of B. chinensis with that of the wild type rhizome of B. chinensis, even though the total amount of flavonoids in the in vitro flavonoid-rich tissue of B. chinensis is far more than that in the wild type rhizome (when either cinnamic acid, which is used as the standard for the flavonoid precursors, or ψ-tectorigenin, which is used as the standard for the isoflavonoid, is used as the standard for the measurement of total flavonoids).

### BRIEF DESCRIPTION OF DRAWINGS

FIG. 1 is a composite of pictures showing the in vitro germination process of B. chinensis on MS0 solid medium. Panel (a), formation of a root on an embryo; Panel (b), elongation of the root and formation of the cotyledon sheath; Panel (c), formation of the first leaf; and Panel (d), formation of the seedlings. Bars=1 cm.

FIG. 2 is a composite showing callus induction and somatic embryos formation in the tissue culture system of B. chinensis. Yellow and transparent calluses were formed on root (a) and leaf (b) explants. Somatic embryos regenerated on soft and watery embryogenic callus(c). Different development stages of somatic embryos were separated (d-i) from embryogenic callus and transplanted to MS0 medium for regeneration. (j) Mature somatic embryos grew on MS0 medium and development process of the somatic embryos to in vitro plantlets was the same as seed germination of wild B. chinensis. (k) many secondary somatic embryos regenerated from base of mature somatic embryo. Bar=1 mm.

FIG. 3 is an HPLC profile of ψ-tectorigenin analyzed by HPLC and detected at the wavelength of 265 nm. ψ-tectorigenin eluted at about 19.5 minutes.

FIG. 4 is an HPLC profile of daidzein analyzed by HPLC and detected at the wavelength of 265 nm. Daidzein eluted at about 16 minutes.

FIG. 5 is an HPLC profile of apigenin analyzed by HPLC and detected at the wavelength of 265 nm. Apigenin eluted at about 21 minutes.

FIG. 6 is an HPLC profile of isorhamnetin analyzed by HPLC and detected at the wavelength of 265 nm. Isorhamnetin eluted at about 21.5 minutes.

FIG. 7 is an HPLC profile of an herbal extract of the rhizome of naturally grown 2-3 year-old B. chinensis used as traditional Chinese medicine. About 0.2 g of the rhizome were ground and extracted with 10 ml of methanol. About 50 μl of the methanol extract were injected to the HPLC.

FIG. 8 is an HPLC profile of an extract taken from the roots of the cultured plantlet of B. chinensis in solid culture containing an MS0 medium. The plantlet was cultivated for about 1 month. About 0.2 g of the roots of the cultured plantlet of B. chinensis were ground and extracted with 10 ml of methanol. About 50 μl of the methanol extract were injected to the HPLC.

FIG. 9 is an HPLC profile of an extract taken from the callus tissue of the B. chinensis in solid culture containing an MS medium with 2 mg/L BA and 2 mg/L NAA. The callus was cultivated for about 5 weeks. About 0.33 g of the callus tissue of the B. chinensis were ground and extracted with 10 ml of methanol. About 50 μl of the methanol extract were injected to the HPLC.

FIG. 10 is an HPLC profile of an extract taken from fast-proliferated roots of the B. chinensi in liquid culture containing an MS medium with 0.1 mg/L BA. The fast-proliferated roots were cultivated for about 5 weeks. About 0.2 g of the fast-proliferated roots of the B. chinensis were ground and extracted with 10 ml of methanol. About 50 μl of the methanol extract were injected to the HPLC.

### DETAILED DESCRIPTION OF THE INVENTION

B. chinensis belongs to the family of Iridaceae, which contains about 300 various plants, many of which have been used in traditional herbal medicines. B. chinensis is well-known for its medicinal use in traditional Chinese herbal medicine. It is better known for its medicinal capability of clearing heat or relieving toxicity, and for improving the condition of the throat (such as for pain and swelling of the throat due to toxin, or phlegm-heat obstruction, and for sore throat). Most of the plants in the Iridaceae family contains flavonoids, such as tectorigenin, irigenin, irisflorentin, dichotomitinn, wogonin, rhamnazin, apocynin, which demonstrated anti-inflammatory, urine secretion, capillary blood vessel diffusion, and anti-viral or bacterial effects.

One aspect of the present invention relates to the establishment of an in vitro flavonoid-rich tissue, which is preferably a callus tissue or fast-proliferated roots, such as an adventitious root, derived from a seed, an embryo of the seed, a root, a leaf, a base of a leaf, or a young inflorescence of B. chinensis in a solid or liquid tissue culture system.

A callus tissue contains a mass of somatic undifferentiated cells from an adult subject plant, which can be made to differentiate into the specialized tissues of a whole plant or a particular part of a plant, such as an adventitious root, with the addition of a number of hormones or enzymes. An adventitious root is a kind of fast-proliferated root. It can be developed from any part other than the radicle. Adventitious roots develop on stems, leaves and even old roots. The radicle or primary root and its lateral roots are the only nonadventitious roots. Many aerial stems naturally form aerial roots.

The in vitro flavonoid-rich tissue is ready for subculture or passage (i.e., where the tissue is at a stationary phase with no visible increase in fresh weight) between 2 to 8 weeks from inoculation, preferably at 4-5 weeks, which is far much shorter than the harvesting of wild type B. chinensis from seeding, which is about 2-3 years. The total flavonoid content in the in vitro flavonoid-rich tissue is much higher than the rhizome of the wild-type B. chinensis, which is well-known for its usage in traditional herbal medicine.

Another aspect of the present invention is to introduce a tissue culture system which establishes the in vitro flavonoid-rich tissue of B. chinensis. The tissue culture system contains a culture medium in either solid (i.e., in agar) or liquid form. In both forms, a salt and a carbohydrate are required. The preferred salt system used in a liquid tissue culture system is a Murashige & Skoog basal medium (“MS salts”), which is named after Murashige & Skoog, Physiol. Plant., 15, 473-497 (1962). The salts contained in the MS basal medium include suitable concentrations of ammonium nitrate, boric acid, calcium chloride, cobalt chloride, cupric sulfate, Na₂-EDTA, ferrous sulfate, magnesium sulfate, manganese sulfate, molybdic acid, potassium iodide, potassium nitrate, potassium phosphate monobasic, sodium nitrate, sodium phosphate monobasic and zinc sulfate.

Examples of other salts that can be used in combination of the MS salts include salts of inorganic acids, such as nitric acid, hydrochloric acid, phosphoric acid, sulfuric acid, boric acid, iodion acid, etc.; organic acids such as acetic acid, malonic acid, mandelic acid, oxalic acid, lactic acid, lactobionic acid, fumaric acid, maleic acid, tartaric acid, citric acid, ascorbic acid, etc.

The carbohydrate used in the culture medium can be any carbohydrate that has nutritional value to a plant culture. Preferably, the carbohydrate is a saccharide. More preferably, the carbohydrate is myo-inositol, sucrose, or a mixture thereof.

Optionally, the culture medium can have a plant growth regulator (PGR). PGRs cause or foster differentiation or dedifferentiation of the explanted tissues being propagated in the culture chamber. Examples of PGRs include, but are not limited to, auxins and cytokinins.

Auxin is the active ingredient in most rooting mixtures. Auxin helps the vegetative propagation of plants. On a cellular level, auxins influence cell elongation, cell division and the formation of adventitious roots. Some auxins are active at extremely low concentrations. Auxins may be used in a concentration range of 0.0001-20 mg/L, preferably 0.01-10 mg/L, more preferably 0.01-2.0 mg/L. Examples of auxins include, but are not limited to, 4-Biphenylacetic acid, 3-Chloro-4-hydroxyphenylacetic acid, 4-Hydroxyphenylacetic acid, Indole-3-acetic acid (IAA), Indole-3-propionic acid, Indole-3-butyric acid, Indole-3-acetyl-L-alanine, Indole-3-acetyl-DL-aspartic acid, Indole-3-acetyl-DL-tryptophan, Indole-3-acetyl-L-valine, 2,4-dichlorophenoxyacetic acid (2,4-D), and alpha-naphthaleneacetic acid (NAA).

Cytokinins promote cell division, stimulate shoot proliferation, active gene expression and metabolic activity in general. At the same time, cytokinins inhibit root formation. This makes cytokinins useful in culturing plant cell tissue where strong growth without root formation is desirable. In addition, cytokinins slow the aging process in plants. Cytokinin may be used in a concentration range of 0.0001-20 mg/L, preferably 0.01-10 mg/L, more preferably 0.01-2.0 mg/L. Examples of cytokinins include, but are not limited to, N-(3-methylbut-2-enyl)-1H-purin-6-amine, 6-benzyl-aminopurine (BA), kinetin, and zeatin.

The culture medium may additionally contain other components such as amino acids, vitamins, or mixtures thereof. Preferred vitamins include, but are not limited to, vitamin B₁ (thiamine HCl), vitamin B₆ (pyridoxine HCl), and vitamin B₃ (nicotinic acid).

The culture medium is adjusted to a pH range suitable for B. chinensis growth. The pH range is preferably between 4 and 8, and more preferably between 5 and 7. In one embodiment, the culture medium includes suitable buffering agents for maintained the pH at the desired level. These agents will typically have a pKa between about 4.5 and about 5.5, and include, but are not limited to, citric acid, N-morpholino-ethansulfonic acid, potassium hydrogen phthalate, and benzoic acid.

The temperature of the culture is usually maintained at or below about 30° C., preferably within the range of about 20-30° C.

A preferred example of the culture medium used in inducing the growth or differentiation of the in vitro flavonoid-rich callus tissue of B. chinensis includes an MS salt, sucrose (30 g/L) and myo-inositol (100 mg/L) (i.e., the MS0 medium). The in vitro flavonoid-rich callus tissue of B. chinensis developed by this culture medium produces the highest flavonoid content.

Another preferred example of the culture medium used in inducing the growth of the in vitro flavonoid-rich callus tissue of B. chinensis includes an MS salt, sucrose (30 g/l), myo-inositol (100 mg/L), and PGRs such as benzyl-aminopurine (BA), 2,4-dichrophenoxy-acetic acid (2,4-D), and/or alpha-naphthaleneacetic acid (NAA). The in vitro flavonoid-rich callus tissue of B. chinensis developed by the liquid MS medium with 0.5 mg/L of NAA produces the highest fresh weight at the stationary phase of the in vitro flavonoid-rich tissue (i.e., at the time when the tissue is ready for subculture where there is no more net fresh weight increase).

A further aspect of the present invention is to provide an analytical means to determine the quantity and types of flavonoids produced in the in vitro tissues of B. chinensis as described above. Flavonoids belong to a class of plant secondary metabolites. They are widely distributed in plants fulfilling many functions including plant defense mechanism, pigmentation and external signaling system.

The total flavonoid content in the in vitro flavonoid-rich tissue of B. chinensis has been determined by measuring the tissue extract in a spectrophotometer at 367 mn using cinnamic acid as a standard. Cinnamic acid is (E)-3-phenyl-2-propenoic acid having the formula of C₆H₅CHCHCOOH. The chemical structure of cinnamic acid is shown below:

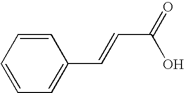

Cinnamic acid is a key intermediate in shikimate and phenylpropanoid pathways. Shikimic acid is a precursor of many alkaloids, flavonoids, aromatic amino acids, and indole derivatives. Phenylpropanoid are a class of plant metabolites based on phenylalanine. Flavonoids are synthesized by the phenylpropanoid metabolic pathway in which the amino acid phenylalanine is used to produce 4-coumaroyl-CoA. Phenylalanine is first converted to cinnamates, coumarines, caffeic acids, ferulic acids, and sinapic acids. Cinnamic acid is the precursor of these acids. These acids can then be combined with malonyl-CoA to yield the true backbone of flavonoids, a group of compounds called chalcones which contain two phenyl rings. Conjugate ring-closure of chalcones results in the familiar form of flavonoids, the three-ringed structure of a flavone. The metabolic pathway continues through a series of enzymatic modifications to yield flavanones→dihydroflavonols→anthocyanins. Along this pathway many products can be formed, including the flavonols, flavan-3-ols, proanthocyanidins (tannins) and a host of other polyphenolics. However, using cinnamic acid as a standard to estimate the total flavonoids in the in vitro tissue can potentially overestimate the total flavonoid content because it tends to include the precursor compounds into the total flavonoid amount.

For this reason, the total flavonoid content is also determined by measuring the tissue extract in a spectrophotomer at 510 nm using ψ-tectorigenin as a standard. ψ-tectorigenin is 5,7-Dihydroxy-3-(4-hydroxyphenyl)-8-methoxy-4H-1-benzopyran-4-one, having the formula of:

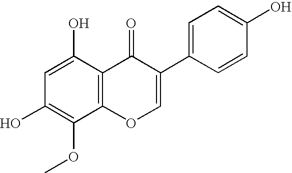

ψ-tectorigenin is an isoflavonoid which is well-known for its antiproliferative effects on cancer cells by steroid receptor signaling. It contains the three ring structure commonly shared by all flavonoids. However, because ψ-tectorigenin is only one of the many flavonoid compounds in the family of flavonoids, using ψ-tectorigenin as a standard tends to underestimate the total flavonoid content in the in vitro tissue.

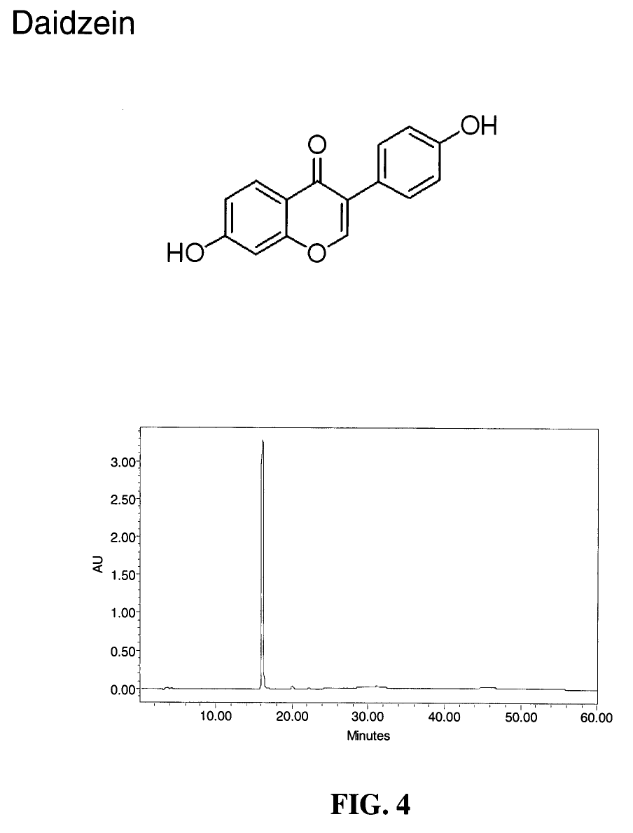

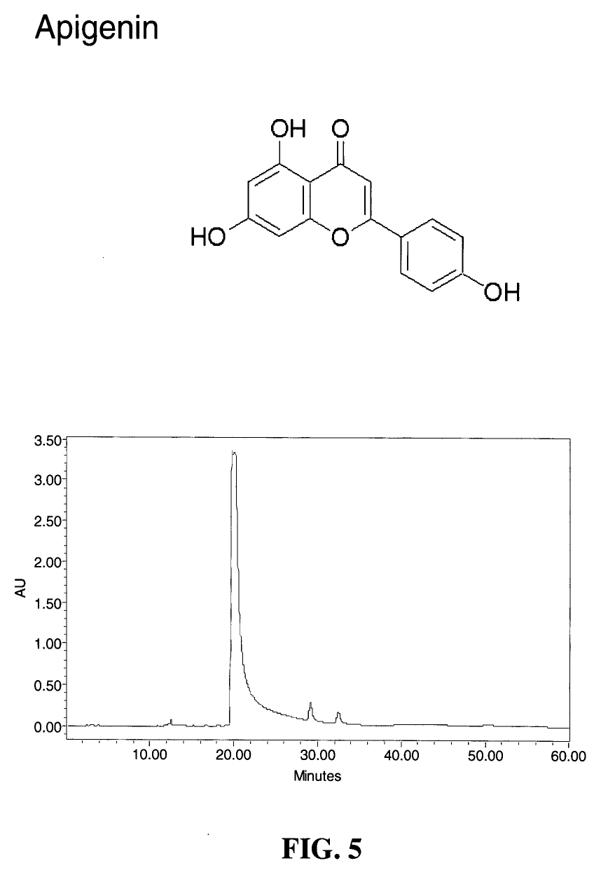

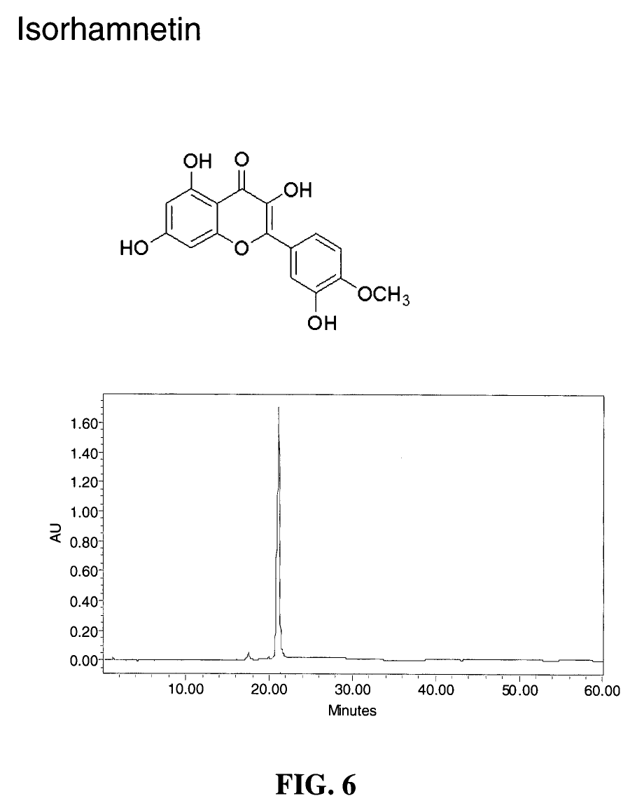

The types of flavonoids produced in the in vitro tissues are further investigated by HPLC analysis in a Water 600 S controller equipped with a Waters™ 626 pump, and Waters 996 Photodiode array detector, using 4 commercially available isoflavonoids, ψ-tectorigenin, daidzein, apigenin, and isorhamnetin, as standards. Each of the isoflavonoid standards is injected into a HYPERSIL-100 C18 (manufactured by Thermo Electron Corporation) column, and the resulting HPLC profile of each of these standards is shown in FIG. 4-6, respectively.

Yet another aspect of the present invention is to provide a method for producing the in vitro flavonoid-rich callus tissue of B. chinensis. This method includes cultivating a B. chinensis tissue which is capable of being propagated in a tissue culture system containing a culture medium which is suitable for growth of the in vitro flavonoid-rich tissue; and growing the B. chinensis tissue in the tissue culture system for a sufficient amount of time to allow the callus tissue to form, which is generally between 2-8 weeks, most likely 4-5 weeks.

The B. chinensis tissue may be any tissue obtained from a naturally grown B. chinensis or a cultured B. chinensis that is capable of proliferation. For example, the in vitro flavonoid-rich B. chinensis tissue can be taken from the a seed, an embryo of the seed, a root, a leaf, a base of a leaf, or a young inflorescence of a naturally grown B. chinensis plant.

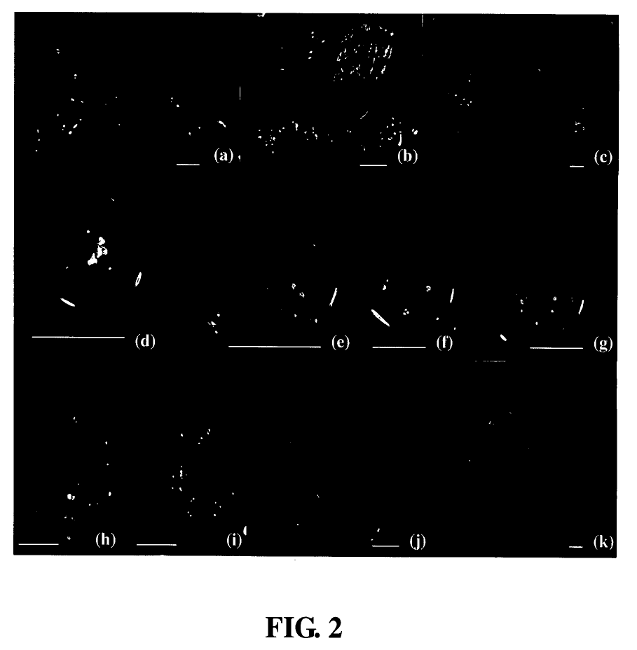

The callus tissue of B. chinensis tissue can be formed on a root explant of B. chinensis in a solid or liquid culture (FIG. 2(a)). The medium may comprise at least one salt and at least one carbohydrate. An example of the formula of the medium contains an MS salt, 30 g/L sucrose, and 100 mg/L myo-inositol (i.e., the MS0 medium). In yet another example, the medium contains an MS salt, 30 g/L sucrose, 100 mg/L myo-inositol and 0.5-2 mg/L BA and/or 0.5-2 mg/L NAA. The callus tissue begins to form about 1 week after the root explant.

The callus tissue of B. chinensis can also be formed from a leaf explant (FIG. 2(b)) in a solid or liquid culture containing an MS medium with or without PGRs. The preferred PGRs include BA and NAA, preferably at 0.5-2 mg/LBA and/or 0.5-2 mg/L NAA. The callus tissue begins to form about 1 week after the leaf explant,

Additionally, the callus tissue of B. chinensis can be formed from somatic embryos (FIG. 2(c)) in a solid or liquid culture containing an MS medium with or without PGRs and undergo different development stages for regeneration (FIG. 2(d)-(j) into an in vitro plantlet similar to a seed germination of wild B. chinensis (FIG. 2(k)).

The callus tissue obtained from the tissue culture system of the B. chinensis tissue capable of proliferation may be further inoculated into another culture medium comprising at least one salt, at least one carbohydrate, and a PGR, such as NAA, and cultured for a desired period of time to be transformed into adventitious roots.

A final aspect of the present invention relates to a method for extracting flavonoids from the in vitro flavonoid-rich callus tissue of B. chinensis. The method includes: (1) drying the tissue; (2) grinding the dried tissue; (3) suspending the ground tissue in an alcohol; (4) heating the suspension; and (5) filtering the suspension to obtain extracts of flavonoids.

The plant tissue may be dried with any method, preferably by freeze-drying using a refrigerated vacuum drier. The alcohol may be any alcohol, preferably methanol or ethanol. The alcohol is preferably heated to a temperature range of 50° C. to 70° C., and more preferably heated with shaking, vibration, or sonication. A preferred method of vibration is ultrasonic wave vibration.

The following experimental designs and result are illustrative, but not limiting the scope of the present invention. Reasonable variations, such as those occur to reasonable artisan, can be made herein without departing from the scope of the present invention. Also, in describing the invention, specific terminology is employed for the sake of clarity. However, the invention is not intended to be limited to the specific terminology so selected. It is to be understood that each specific element includes all technical equivalents which operate in a similar manner to accomplish a similar purpose.

### EXAMPLES

### Example 1

### Material and Methods

### Culture Medium

The liquid culture medium comprised an MS basal medium (which contains the salts of Table 1, infra) or an MS0 medium (which contains the salts of Table 1, 1 mg/L thiamine HCl [vitamin B1], 0.5 mg/L pyridoxine HCl [vitamin B6], 0.5 mg/L nicotinic acid [vitamin B3], 2 mg/L glycine, 100 mg/L myo-inositol, and 30 g/L sucrose) and/or various plant growth factors according to different experiments. The solid culture medium was prepared by adding 8 g/L agar to the liquid medium. The solid culture was carried out in 8×1.5×1.5 cm culture tubes (10 ml culture medium/tube) or flasks (30 ml culture medium/flask). Liquid culture was carried out in 50 ml, 100 ml or 250 ml flasks with 10 ml, 50 ml or 100 ml culture medium, respectively. The opening of the culture tubes or flasks was covered with aluminum foil.

All the culture medium had a pH value of 5.70 0.05, and were sterilized by autoclaving at 121° C., under 1.1-1.2 kg/cm² pressure, for 15 minutes.

TABLE 1
The Salts Composition of the MS Basal Medium
(Murashige and Skoog, 1962)

| Chemical | mg/L |
|---|---|
| Macronutrients | |
| KNO₃ | 1900 |
| NH₄NO₃ | 1650 |
| MgSO₄7H₂O | 370 |
| CaCl₂2H₂O | 440 |
| KH₂PO₄ | 170 |
| Micronutrients | |
| MnSO₄4H₂O | 22.3 |
| KI | 0.83 |
| H₃BO₃ | 0.2 |
| ZnSO₄7H₂O | 8.6 |
| CuSO₄5H₂O | 0.025 |
| Na₂MoO₄2H₂O | 0.25 |
| CoCl₂6H₂O | 0.025 |
| FeSO₄7H₂O | 27.8 |
| Na₂EDTA | 37.3 |

B. chinensi Tissues

Fruits of B. chinensis were collected in Yuang Shan Station Park in December 2005.

The B. chinensis fruits were placed in 2% sodium hypochloride solution with two drops of Tween 20, washed and disinfected for 5 minutes by ultrasonic wave, then washed three times with sterile distilled water in laminar flow hood. The fruit pod was opened and the seeds were taken out. The seeds were sliced into two halves under a stero microscope and the embryo were removed from the seeds and cultured in the MS0 medium (see Table 2). B. chinensis seedling were formed two weeks later. Wild B. chinensis samples were purchased from Benchiao Chan-Chun Pharmacy for Traditional Chinese Medicine in Taipei, Taiwan.

### Callus Induction and Somatic Embryogenesis

Two week old B. chinensis seedlings were dissected into leaf, leaf base and root sections, cut into fragments having a length of about 5 mm, and cultured in callus induction medium (MS basal medium supplemented with various amount of BA and/or mg/l NAA, see Table 2, infra) with light or in the dark.

TABLE 2
Composition of Callus Induction Media

| | | | | | | | Plant Growth | | | |
|---|---|---|---|---|---|---|---|---|---|---|
| | | | | | | | Regulator | | | |
| Test | Mineral | Organic Substance (mg/L) | | | | | (mg/L) | | Sucrose | |
| sample | Comp. | V_{B1} | V_{B6} | V_{B3} | Gly | MI | NAA | BA | (g/L) | pH |
| MSO | MS | 1 | 0.5 | 0.5 | 2 | 100 | 0 | 0 | 30 | 5.7 |
| BC1 | MS | 1 | 0.5 | 0.5 | 2 | 100 | 1 | 0 | 30 | 5.7 |
| BC2 | MS | 1 | 0.5 | 0.5 | 2 | 100 | 2 | 0 | 30 | 5.7 |
| BC3 | MS | 1 | 0.5 | 0.5 | 2 | 100 | 0 | 1 | 30 | 5.7 |
| BC4 | MS | 1 | 0.5 | 0.5 | 2 | 100 | 1 | 1 | 30 | 5.7 |
| BC5 | MS | 1 | 0.5 | 0.5 | 2 | 100 | 2 | 1 | 30 | 5.7 |
| BC6 | MS | 1 | 0.5 | 0.5 | 2 | 100 | 0 | 2 | 30 | 5.7 |
| BC7 | MS | 1 | 0.5 | 0.5 | 2 | 100 | 1 | 2 | 30 | 5.7 |
| BC8 | MS | 1 | 0.5 | 0.5 | 2 | 100 | 2 | 2 | 30 | 5.7 |

V_{B1}: thiamine HCl,
V_{B6}: pyridoxine HCl,
V_{B3}: nicotinic acid,
Gly: glycine,
MI: myo-inositol

About 0.3 g of B. chinensis callus tissue, induced with the a callus induction medium containing 2 mg/L NAA (sample BC8, Table 2, infra), was inoculated in 50 ml flask containing 10 ml liquid culture medium supplemented with various amount of NAA and/or BA (Table 3). The fresh weight and total flavonoid were measured four weeks later. The profile of the flavonoids four weeks after the tissue was induced was analyzed by spectrophotometer using cinnamic acid as a standard.

The B. chinensis seedling cultured in solid MS0 medium could be subcultured about every 8 weeks, each producing 2-3 new buds. The B. chinensis seedling cultured in liquid MS0 medium could be subcultured about every 5 weeks, each producing 4-5 new buds. The B. chinensis callus tissue cultured in liquid MS medium with 0.5 mg/L of NAA had about 3.78 folds of fresh weight increase in about 4 weeks after the culture. The fast-proliferated roots of B. chinensis formed from rhizome tissue of B. chinensis by liquid culture containing MS medium and 0.1 mg/L BA could be subcultured about every 3 weeks, each with about 1.2 times of fresh weight increase. Also, after 2 passages, the fresh weight of the fast-proliferated roots of B. chinensis could be increased to about 2.37 folds.

TABLE 3
Composition of Flask Culture Medium

| | | | | | | | Plant Growth | | | | |
|---|---|---|---|---|---|---|---|---|---|---|---|
| Test | Mineral | Organic Substance (mg/L) | | | | | Regulator (mg/L) | | | Sucrose | |
| sample | Comp. | V_{B1} | V_{B6} | V_{B3} | Gly | MI | NAA | 2,4-D | Kinetin | (g/L) | pH |
| BL1 | MS | 1 | 0.5 | 0.5 | 2 | 100 | 0 | 0 | 0 | 30 | 5.7 |
| BL2 | MS | 1 | 0.5 | 0.5 | 2 | 100 | 0.5 | 0 | 1.0 | 30 | 5.7 |
| BL3 | MS | 1 | 0.5 | 0.5 | 2 | 100 | 1.0 | 0 | 0 | 30 | 5.7 |
| BL4 | MS | 1 | 0.5 | 0.5 | 2 | 100 | 0 | 0.5 | 0 | 30 | 5.7 |
| BL5 | MS | 1 | 0.5 | 0.5 | 2 | 100 | 0.5 | 0.5 | 0 | 30 | 5.7 |
| BL6 | MS | 1 | 0.5 | 0.5 | 2 | 100 | 1.0 | 0.5 | 0 | 30 | 5.7 |
| BL7 | MS | 1 | 0.5 | 0.5 | 2 | 100 | 0 | 1.0 | 0 | 30 | 5.7 |
| BL8 | MS | 1 | 0.5 | 0.5 | 2 | 100 | 0.5 | 1.0 | 0 | 30 | 5.7 |
| BL9 | MS | 1 | 0.5 | 0.5 | 2 | 100 | 1.0 | 1.0 | 0 | 30 | 5.7 |

V_{B1}: thiamine HCl,
V_{B6}: pyridoxine HCl,
V_{B3}: nicotinic acid,
Gly: glycine,
MI: myo-inositol

Extraction of in vitro Flavonoid-Rich Tissue for Flavonoids and Total Flavonoid Analysis

The in vitro flavonoid-rich tissue of B. chinensis was frozen at −80° C. for 10 hours, dried in a refrigerated vacuum drier (VirTis Freezemobile 12XL) overnight, and ground. An adequate quantity of dried and ground tissue was suspended in an adequate amount of methanol in a 10 ml flask, incubated at 60° C. with ultrasonic vibration for about 1 hour, and cooled to room temperature. Methanol was then added to the suspension to a final volume of 10 ml. The suspension was filtered with Whatman No. 1 filter paper. The filtered solution was sealed in brown sample vials and placed in cold room for future analysis.

### Measurement of Total Flavonoid Using Cinnamic Acid as a Standard

About 1.5 ml of B. chinensis extract was added to 1.5 ml of 10% AlCl₃, thoroughly mixed and incubated for 10 minutes. The absorption at 367 nm was determined by a spectrophotometer. A standard curve was generated using the precursor of flavonoid, cinnamic acid (ALDRICH, trans-cinnamic acid Cat No. C8, 085-7)

### Measurement of the Total Flavonoid Using ψ-Tectorigenin as a Standard

Total flavonoid was measured according to Lee et al., J. Agric. Food Chem. (2003) 51: 7292-7295. Briefly, 4 ml of deionized water and 0.3 ml of 5% NaNO₂ were added to about 0.5 ml of B. chinensis extract to form a sample mixture and allowed for reaction for about 5 minutes. About 0.3 ml of 10% AlCl₃ was then added to the reacted sample mixture and allowed for further reaction for an additional 5 minutes under thorough shaking. This was followed by the addition of 2 ml of 1 N NaOH and 2.9 ml of deionized water to the further reacted sample mixture. The total flavonoid in the resulting reacted sample mixture was then determined by measuring the absorbance at 510 nm wavelength with an UV spectrophotometer (Ultrospec 2000, Pharmacia Biotech), and comparing the data with the standard curve using ψ-Tectorigenin (Sigama T-9165, ψ-tectorigenin Lot No. 072K16451) as a standard.

### Analysis of Flavonoids by HPLC Using Tectorigenin, Daidzein, Apigenin, and Isorhamnetin as Standards

The various types of flavonoids found in the in vitro flavonoid-rich tissues of B. chinensis were analyzed by HPLC using a HYPERSIL-100 C18 column (Thermo Electron Corporation), a Degasser ( ERC-3415α), a Waters 600Scontroller, a Waters™ 626 pump, and a Waters 996 Photodiode Array Detector. The analysis was performed using a gradient containing Solution A: 75% H₂O, 19% acetonitirle, 5% methanol, and 1% THF; and Solution B: 55% acetonitrile, 15% methanol, and 30% H₂O in the condition set forth in Table 4:

TABLE 4
HPLC Gradient for Analysis of Tectorigenin,
Daidzein, Apigenin, and Isorhamnetin

| Time | Solution A | Solution B |
|---|---|---|
| 0 | 98 | 2 |
| 5 | 98 | 2 |
| 10 | 72 | 28 |
| 15 | 50 | 50 |
| 20 | 50 | 50 |
| 25 | 40 | 60 |
| 40 | 40 | 60 |
| 45 | 20 | 80 |
| 50 | 20 | 80 |
| 60 | 98 | 2 |

The flow rate was 1 ml/min. The wavelength was between 200 and 800 nm with the detection wavelength of 265 nm.

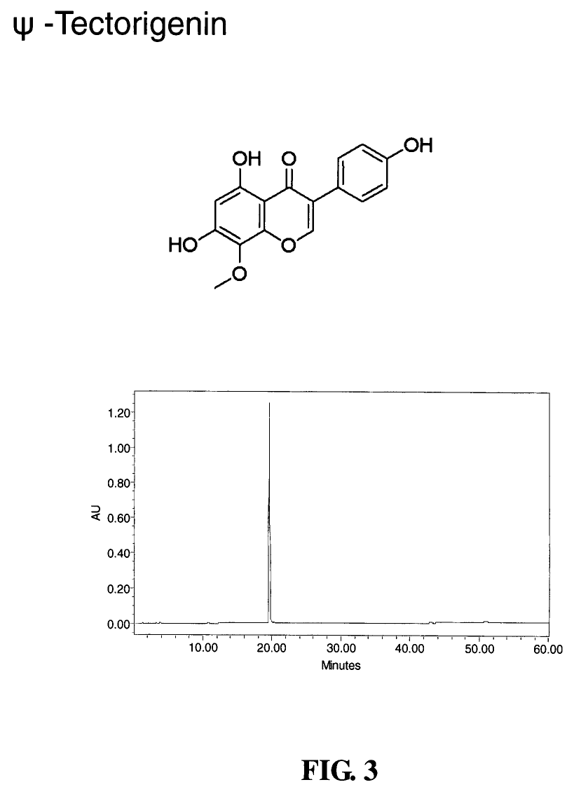

As shown in FIGS. 3-6, tectorigenin, daidzein, apigenin, and isorhamnetin were eluted from the gradient between 16-22 minutes.

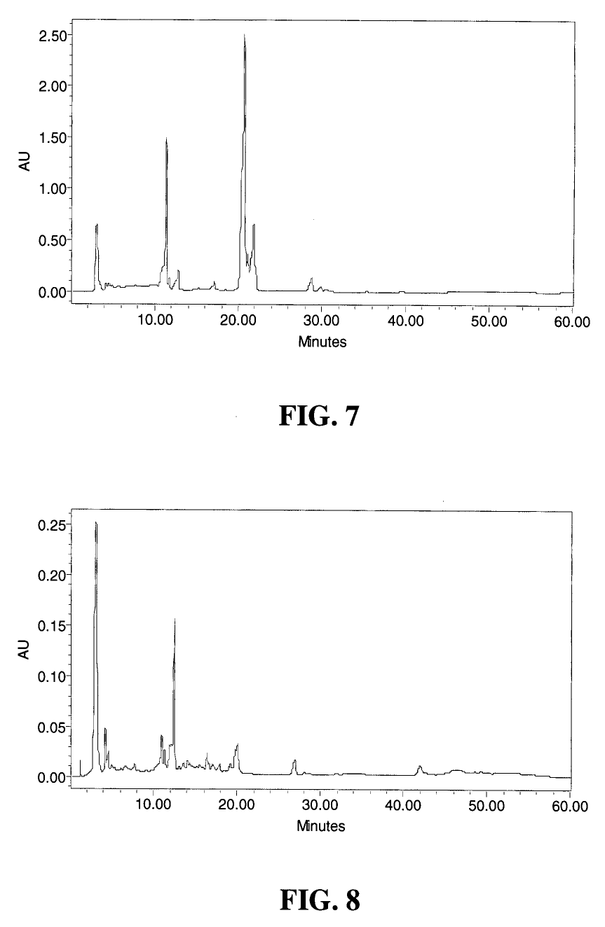

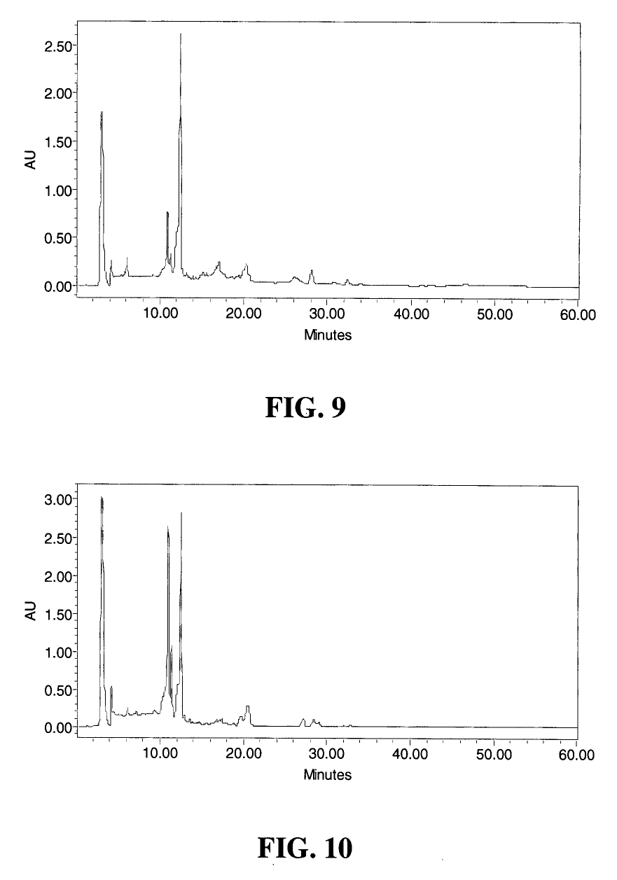

The samples included (1) the naturally grown rhizome tissue of B. chinensis (FIG. 7), (2) the roots of a B. chinensis plantlet cultured in solid MS0 medium (FIG. 8); (3) the callus of B. chinensis cultured in solid MS medium with 2 mg/L BA and 2 mg/L NAA (FIG. 9); and (4) the fast-proliferated roots of B. chinensis cultured in liquid MS medium with 0.1 mg/L BA (FIG. 10). Other than the callus tissue, where 0.33 g of the tissue were extracted, 0.2 g of the tissue were extracted in 10 ml of methanol, in which 50 μl were injected into the column for HPLC analysis. Also, other than the naturally grown rhizome tissue, which was grown in the wild for about 2-3 years, the rest of the tissues were about 4-5 weeks old.

### Statistic Analysis

All the measurements were repeated for three times. The experimental data were analyzed using Duncan's multiple range test with 5% significance level (Duncan, D. B. 1955, Biometrics, 11:1-42).

### Morphology Observation and Photograph

Morphology observations were made under a stereo microscope (Olympus SZ-ET). Photographic records were made using a digit camera (Nikon coolpix 8700).

### Example 2

### Establishment of in Vitro Culture

### From Seeds

B. chinensi seeds were cut into two halves and placed on solid MS0 medium (see Table 2) or MS0 supplemented with 2 mg/L BA with the cross section facing down to the solid medium. Germination may occur within one week. On the medium supplemented with BA, the seedling usually had 2 or 3 buds, which does not occur on the MS0 medium.

### From Embryo

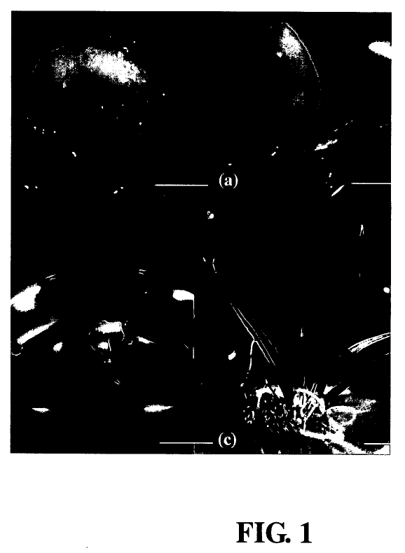

Embryos were removed from B. chinensis seeds under a dissecting microscope, and cultured on solid MS0 medium. The embryo gradually turned into a green color after three days in culture (FIG. 1, panel (a) and (b)). Seedlings were formed after 14 days (FIG. 1, panel (c)). Panel (d) of FIG. 1 shows seedlings of B. chinensis after 8 weeks on MS0 culture medium.

### Example 3

### Induction of Callus on Different Part of Seedling and Somatic Embryogenesis

B. chinensi seedling was divided into three sections: root, leaf base and leaf, and tested for callus induction. Among the three sections, the root provided the best response to callus induction. Callus tissue formed on the root as long as the culture medium contained BA or NAA. With an MS basal medium supplemented with 2 mg/L NAA and 2 mg/L 2,4-D, yellow callus tissue could form in 7 days (FIG. 2, panel (a)). In contrast, it took 14 days for the callus tissue to form on leaf cultured in medium supplemented with 1-2 mg/L BA and 0.2 mg/L NAA.

The callus tissue formed upon induction may be divided into two types. The callus tissue induced by 1 mg/L BA, 1 mg/L BA and 1 mg/L NAA, 1 mg/L NAA resembled the root tissue with a white color and root hair (root-like callus). After passage in medium containing 2 mg/L BA and 2 mg/L NAA, the color turned yellow but the root-like morphology remained unchanged. The callus tissue formed in medium containing 2 mg/L BA and 2 mg/L NAA, or 1 mg/L BA and 2 mg/L NAA after two months of culture in dark or dim light had a dark yellow color and formed somatic embryos (FIG. 2, panels (d)-(k)). Comparison of the development of the somatic embryos and the sexual embryos from the seed culture (FIG. 1) showed no morphological difference.

### Example 4

### Flask Culture of B. chinensis

Root-like callus tissue induced by MS medium with 2 mg/L BA and 2 mg/L NAA was inoculated at an inoculum weight of 3% (0.3 g F.W./10 ml culture medium) in culture media containing various amount of NAA and 2,4-D (Table 5). The tissue weight increased in the medium containing 0.5 mg/L or 1.01 mg/L NAA, as well as in the medium containing 0.5 mg/L 2,4-D. After 4 weeks, the callus tissue cultured in the medium containing 0.5 mg/L NAA showed the highest fresh weight of 1.435±0.082 g and formation of adventitious roots. The callus tissue did not survive in the other six media (i.e., media that contain no NAA or 1 mg/L NAA).

### Example 5

### Total Flavonoid Content in Cultured and Wild B.chinensis Tissue

The total flavonoid contents using cinnamic acid as a standard were listed in Table 5. The highest flavonoid content (2713±88.15 mg cinnamic acid eq./g dry weight) was obtained in the callus tissue cultured in the MS0 medium without the addition of the PGRs such as 2,4-D and/or NAA.

TABLE 5
Total Flavonoid Content in B. chinensis Callus Tissue
Cultured with Different Amount of PGRs

| | |
|---|---|
| | Total flavonoid |
| PGR (mg/L) | (mg cinnamic acid |

| 2,4-D | NAA | Growth rate¹ | eq./g D.W.) |
|---|---|---|---|
| 0 | 0 | 0.85 0.10² cd³ | 2713.4816 88.15 a |
| 0 | 0.5 | 3.78 0.27 a | 1992.5359 27.11 b |
| 0 | 1 | 2.41 0.35 b | 1608.1228 32.67 bcd |
| 0.5 | 0 | 1.23 0.09 c | 862.2292 24.10 e |
| 1.0 | 0 | 0.27 0.21 e | 1193.3614 94.30 de |
| 0.5 | 0.5 | 0.54 ± 0.31 de | 1309.6969 ± 41.28 cde |
| 0.5 | 1.0 | 0.20 ± 0.15 e | 1137.7226 ± 91.60 de |
| 1.0 | 0.5 | 0.54 ± 0.25 de | 1168.7844 ± 63.81 de |
| 1.0 | 1.0 | 0.26 ± 0.07 e | 1319.8130 ± 35.62 cde |

¹Growth rate = (Final F.W. − Initial F.W.)/Initial F.W..
²Values are means of 3 replicates ± S.E.
³Means within a column followed by the same (a to e) letters are not significantly different by Duncan's multiple range test (P > 0.05).

The total flavonoid contents of the callus tissue using ψ-Tectorigenin as a standard were listed in Table 6. The highest flavonoid content (34.667±1.050 mg ψ-tectorigenin eq./g dry weight) was obtained in the callus tissue cultured in the liquid culture containing MS medium with 0.5 mg/L NAA.

TABLE 6
Total Flavonoid Content in B. chinensis Callus Tissue Using ψ-
Tectorigenin As a Standard

| Culture | Regenerated | PGRs | Total flavonoids* |
|---|---|---|---|
| type | tissues | (mg/L) | (Eq. ψ-tectorigenin mg/g DW) |
| Solid | callus | BA (0.5) | 28.468 ± 6.809 |
| Culture | | NAA (0.5) | |
| Liquid | callus | NAA (0.5) | 34.667 ± 1.050 |
| Culture | | | |

| | |
|---|---|
| Rhizome of Wild-Type | 11.500 ± 0.1 |
| B. chinensis | |

*Values are means of 3 replicates ± S.E.

As shown in Table 6, the callus tissue in either the solid or liquid culture contained about 3 times of the total flavonoids when ψ-Tectorigenin was used as a standard than that of the wild-type B. chinensis.

As shown in Table 7, the total flavonoid content in cultured B. chinensis callus tissue (2713.48±88.15 mg cinnamic acid eq./g D.W.) was much higher than that of the wild rhizome tissue (337.70±15.05 mg cinnamic acid eq./g D.W.). Moreover, the normal growing period of wild rhizome tissue is 2-3 years, while the culturing period for the callus tissue was only 2 to 8 weeks. Accordingly, the present invention clearly provides a more cost effective method for producing total flavonoid from B. chinensis.

TABLE 7
The Total Flavonoid Content in Cultured Callus and Wild
rhizomes of B. chinensis.

| | Total flavonoid² |
|---|---|
| Belamcanda chinensis | (mg cinnamic acid eq./g D.W.) |
| Callus (Flask culture¹) | 2713.48 ± 88.15 |
| Rhizome (Wild) | 337.70 ± 15.05 |

¹The callus culture medium was MS without PGRs.
²Values are means of 3 replicates ± S.E.

The HPLC profile analysis of the flavonoids from (1) the naturally grown rhizome tissue of B. chinensis; (2) the roots of a B. chinensis plantlet cultured in solid MS0 medium; (3) the callus of B. chinensis cultured in solid MS medium with 2 mg/L BA and 2 mg/L NAA; and (4) the fast-proliferated roots of B. chinensis cultured in liquid MS medium with 0.1 mg/L BA, are shown in FIGS. 7-10.

As shown in FIGS. 3-6, tectorigenin, daidzein, apigenin, and isorhamnetin were eluted from the gradient between 16-22 minutes, which represent some of the commercially available isoflavonoids. However, isoflavonoids, which is derived from 3-phenylbenzopyrone, is just one of the many different types of flavonoids found in the plant, such as chalcones, flavanones, flavones, flavonols, anthocyanidins (flavylium cations), flavan 3-ols (catechins), flavan 3,4-diols (proanthocyanidins), biflavonoids and oligomeric flavonoids, and the aurones, which are primarily different from the oxidation level or substitution pattern of the heterocyclic ring. At this time, more than 1300 different flavonoid compounds have been reported to be isolated from plants.

As shown in FIGS. 7, the naturally grown rhizome of B. chinensis contained a fair amount of compounds eluted from the column at about 20-22 minutes, and detected at 265 nm. These compounds corresponded to the isoflavonoid standards fairly well, and should presumably belong to various types of isoflavonoids. There were two other significant peaks shown in the HPLC profile of the naturally grown rhizome, which were peaked at 2-4 minutes and 10-13 minutes. These compounds corresponded to the cinnamic acid fairly well, and should presumably belong to various precursors of the flavonoids. Trans-cinnamic acid eluted from the HPLC column at about 10 minutes.

The roots of the plantlet (FIG. 8), the callus tissue (FIG. 9) and the fast-proliferated roots (FIG. 10) all showed a small but broad peak which spanned from about 16 to 21 minutes, but a much more significant peak at 10-13 minutes. This indicated that although the in vitro tissues were far much younger than that of the naturally grown tissue, they essentially contained the similar flavonoids. This further confirmed, based on the findings in Tables 6 and 7, that the majority of the elevated amount of flavonoids found in the in vitro tissues corresponded to the precursors of flavonoids, as represented by trans-cinnamic acid, even though the total amount of isoflavoids, as determined by ψ-tectorigenin, also increased substantially (i.e., about 3 times as compared to that in the rhizome of the wild-type B. chinensis).

The embodiments illustrated and discussed in this specification are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

1. An in vitro flavonoid-rich tissue of Belamcanda chinensis produced in a tissue culture system, wherein said in vitro flavonoid-rich tissue of Belamcanda chinensis is a root of a plantlet, a callus tissue, or a fast-proliferated root, wherein said in vitro flavonoid-rich tissue of Belamcanda chinensis contains elevated amount of flavonoids than a wild type rhizome tissue of Belamcanda chinensis, and wherein said in vitro flavonoid-rich tissue is suitable for subculture in about 2 to 8 weeks.

2. (canceled)

3. The in vitro flavonoid-rich tissue of B. chinensis according to claim 2, wherein said fast-proliferated root is formed from said callus tissue or said root of said plantlet.

4. The in vitro flavonoid-rich tissue of B. chinensis according to claim 1, wherein said in vitro flavonoid-rich tissue is ready for subculture in about 4-5 weeks.

5. The in vitro flavonoid-rich tissue of B. chinensis according to claim 1, wherein said in vitro flavonoids-rich tissue is cultured from a B. chinensis tissue capable of proliferation.

6. The in vitro flavonoid-rich tissue from B. chinensis according to claim 5, wherein said B. chinensis tissue capable of proliferation is a seed, an embryo of said seed, a root, a leaf, a base of a leaf, or a young inflorescence of B. chinensis.

7. The in vitro flavonoid-rich tissue from B. chinensis according to claim 1, wherein said tissue culture system comprises a culture medium which comprises a salt medium and a carbohydrate.

8. The in vitro flavonoid-rich tissue from B. chinensis according to claim 7, wherein said salt medium is a Murashige and Skoog basic salt medium (MS medium) which comprises sodium, potassium, nitrate, ammonium, magnesium, sulfate, calcium, iron, chloride, phosphate, manganese, iodine, borate, zinc, copper, molybdenum, cobalt, or a mixture thereof.

9. The in vitro flavonoid-rich tissue from B. chinensis according to claim 7, wherein said carbohydrate is myo-inositol or sucrose or a mixture thereof.

10. The in vitro flavonoid-rich tissue from B. chinensis according to claim 7, wherein said culture medium further comprises a plant growth regulator, which is at least one selected from the group consisting of indole-3-acetic acid (IAA), 2-4- dichlorophenoxyacetic acid (2,4-D), α-naphthaleneacetic acid (NAA), 6-benzyl-aminopurine (BA), and kinetin.

11. The in vitro flavonoid-rich tissue from B. chinensis according to claim 7, wherein said culture medium further comprises a vitamin, which is at least one selected from the group consisting of thiamine HCl, pyridoxine HCl, and nicotinic acid.

12. The in vitro flavonoid-rich tissue from B. chinensis according to claim 7, wherein said culture medium is at a pH of about 5.0 to 7.0.

13. The in vitro flavonoid-rich tissue from B. chinensis according to claim 1, wherein said tissue culture system is a solid or liquid flask culture.

14. The in vitro flavonoid-rich tissue from B. chinensis according to claim 2, wherein said total flavonoid content of said callus tissue cultured in said tissue culture system containing an MS salt is about 3 times of said rhizome of a wild-type B. chinensis when ψ-tectorigenin is used as a standard.

15. The in vitro flavonoid-rich tissue from B. chinensis according to claim 2, wherein said total flavonoid content of said callus tissue cultured in said tissue culture system containing an MS medium with one or more plant growth regulator is about 9 times of said rhizome of a wild-type B. chinensis when cinnamic acid is used as a standard.

16. A method for producing the in vitro flavonoid-rich tissue of B. chinensis according to claim 1, comprising:
inoculating a B. chinensis tissue in said tissue culture system; wherein said B. chinensis tissue is capable of proliferation; wherein said tissue culture system contains a culture medium which is suitable for growth of said in vitro flavonoid-rich tissue and
growing said B. chinensis tissue in said tissue culture system for a sufficient amount of time to allow said callus tissue to be ready for subculture.

17. The method according to claim 16, wherein said culture medium is maintained at about 20° C. to 30° C.

18. The method according to claim 16, wherein said B. chinensis tissue is a seed, an embryo of said seed, a root, a leaf, a base of a leaf, or a young inflorescence of B. chinensis.

19. The method according to claim 16, wherein said tissue culture system is a flask culture.

20. The method according to claim 16, wherein said sufficient amount of time is about 2 to 8 weeks.

21. The method according to claim 16, wherein said time for subculture is about 4-5 weeks.

22. The method according to claim 16, wherein said culture medium comprises a salt medium, and a carbohydrate.

23. The method according to claim 22, wherein said salt medium is a Murashige and Skoog basic salt medium (MS medium) which comprises sodium, potassium, nitrate, ammonium, magnesium, sulfate, calcium, iron, chloride, phosphate, manganese, iodine, borate, zinc, copper, molybdenum, cobalt, or a mixture thereof.

24. The method according to claim 22, wherein said carbohydrate is myo-inositol or sucrose or a mixture thereof.

25. The method according to claim 22, wherein said culture medium further comprises a plant growth regulator which is at least one selected from the group consisting of indole-3-acetic acid, 2-4-dichlorophenoxyacetic acid, α-naphthaleneacetic acid, 6- benzyl-aminopurine, and kinetin.

26. The method according to claim 22, wherein said culture medium further comprises a vitamin which is at least one selected from the group consisting of thiamine HCl, pyridoxine HCl, and nicotinic acid.

27. The method according to claim 22, wherein said culture medium has a pH of about 5 to 7.

28. A method for extracting flavonoids from said in vitro flavonoid-rich tissue of B. chinensis according to claim 1, comprising:
drying said in vitro flavonoid-rich tissue of B. chinensis to obtain a dried flavonoid-rich tissue;
grinding said dried flavonoid-rich tissue;
adding an alcohol to said dried flavonoid-rich tissue to form a suspension;
heating said suspension to form a heated suspension; and
filtering said heated suspension after said heated suspension has cooled off to collect an filtrate which contains said flavonoids.

29. The method according to claim 28, wherein said dried flavonoid-rich tissue is obtained by subjecting said flavonoid-rich tissue to freeze-drying.

30. The method according to claim 28, wherein said suspension is heated at about 50-70° C.

31. The method according to claim 28, wherein said suspension is heated with vibration.

32. The method according to claim 31, wherein said vibration is generated by an ultrasonic wave.

33. The method according to claim 28, wherein said alcohol is methanol or ethanol.

34. The method according to claim 28, wherein said filtrate is collected by passing said heated suspension through a Whatman® No. 1 filter.

35. A method for determining a total amount of said flavonoids extracted from said in vitro flavoid-rich tissue of B. chinensis according to claim 28, comprising:
measuring said filtrate in a spectrophotometer at an absorbance at 367 nm wavelength to obtain a sample absorbance value; and
comparing said sample absorbance value to a standard absorbance value using a known amount of cinnamic acid.

36. The method according to claim 35, wherein said filtrate is pretreated with an adequate amount of AlCl₃ prior to the measurement of the absorbance at 367 nm.

37. A method for determining a total amount of said flavonoids extracted from said in vitro flavoid-rich tissue of B. chinensis according to claim 28, comprising:
measuring said filtrate in a spectrophotometer at an absorbance of 510 nm wavelength to obtain a sample absorbance value; and
comparing said sample absorbance value to a standard absorbance value using a known amount of ψ-tectorigenin as a standard.

38. The method according to claim 36, wherein said filtrate is pretreated with an adequate amount of NaNO₂ and AlCl₃ prior to the measurement of the absorbance at 510 nm.

39. A method for determining various types of said flavonoids extracted from said in vitro flavoid-rich tissue of B. chinensis according to claim 28, comprising:
injecting said filtrate to a column of a high performance liquid chromatography (HPLC); and
recording the elution profile of said HPLC at an absorbance of 265 nm.
